(19) United States
(12) Patent Application Publication
(10) Pub. No.: US 2016/0089422 A1
(43) Pub. Date: Mar. 31, 2016

(54) METHOD FOR INACTIVATING A PRION PROTEIN

(71) Applicant: LABORATOIRE FRANCAIS DU FRACTIONNEMENT ET DES BIOTECHNOLOGIES, Les Ulis (FR)

(72) Inventors: Abdessatar Sami Chtourou, Elancourt (FR); Benoît Flan, Limours (FR)

(21) Appl. No.: 14/891,139

(22) PCT Filed: May 15, 2014
(86) PCT No.: PCT/EP2014/060014

§ 371 Date: Nov. 13, 2015

(30) Foreign Application Priority Data
May 15, 2013 (FR) 1354369

(51) Int. Cl. A61K 38/44; A61K 35/14

(52) U.S. Cl. CPC A61K 38/44; A61K 35/14

(57) ABSTRACT

The invention relates to a process for inactivating pathological prion proteins (PrPsc) in a sample or a material which may be contaminated by a pathological prion protein, wherein the sample or the material which may be contaminated by a pathological prion protein is put into contact with at least one methionine sulfoxide reductase.

The present invention relates to a process for inactivating pathological prion proteins in a biological product or in a materiel which may be contaminated by a pathological prion protein. More particularly, the invention relates to a process for biological inactivation, using a methionine sulfoxide reductase (MSR).

### TECHNOLOGICAL BACKGROUND

Transmissible spongiform encephalopathies (TSEs), also called prion diseases, are degenerative diseases of the central nervous system (CNS) which affect both humans (for example Creutzfeldt-Jakob disease (CJD) and kuru) and animals (notably ovine scrapies and bovine spongiform encephalopathy).

The etiological agent of these diseases is classified in the category of “Unconventional Transmissible Agents” (UTAs). The protein of the prion in its pathological form called PrPsc, represents the marker of prion diseases. This protein is presently considered as the element bearing infectiosity found in every case of infection with UTAs, in particular in the brain. The PrPsc is capable of causing symptoms of TSEs by intracerebral inoculation in a healthy individual. Indeed, the agent responsible for the replication or propagation of PrPsc would be the pathological prion protein itself because it is capable of propagating or multiplying exponentially, by deforming the sound prion proteins into pathological prion proteins. The so-called “pathological” form of PrP is therefore the protein form, the conformation of which is correlated with the occurrence of TSE in human or non-human infected animals.

The modified three-dimensional structure of the PrPsc as compared with that of the PrPc, gives it atypical physico-chemical properties, which are expressed by greater resistance to usual disinfection and sterilization means (heat, chemicals, enzymes, etc.).

Further, many investigations aiming at detecting the presence of blood infectiosity in TSEs have been conducted these recent years (ref: Brown, P., Vox Sanguinis (2005) 89, 63-70). The whole of this work shows that very low blood infectiosity (10-30 Infectious Units/ml) may be experimentally demonstrated in certain prion diseases.

The use in the pharmaceutical industry of blood derivatives, such as plasma proteins from coagulation, has been experiencing a continuous growth for many years. And the precaution principle associated with the use of such products implies that a procedure be systematically set into place for at best guaranteeing the removal and/or the inactivation of the possible pathological prion proteins in these products.

Known processes for removing prion proteins most often resort to retention and/or filtration means, which may sometimes lead to concomitant removal and more or less significant removal of plasma proteins of interest.

Therefore there exists a real need for a process allowing decontamination of a biological product and notably a blood product or a derivative of blood, in relation to pathological prion proteins, which does not impoverish said biological product.

### SUMMARY OF THE INVENTION

In this context, the inventors propose the use of a particular enzyme, i.e. methionine sulfoxide reductase (MSR), for treating a biological product and inactivating the potentially present pathological prion proteins. MSR according to the invention may also be used for treating a piece of equipment, such as a surgical instrument, an inert or other surface.

Therefore the object of the invention is a process for inactivating pathological prion proteins (PrPsc) in a sample or a material which may be contaminated with a pathological prion protein, wherein the sample or the material which may be contaminated with a pathological prion protein, is put into contact with at least one methionine sulfoxide reductase (MSR).

In a particular embodiment, the MSR is immobilized, preferentially with a covalent bond, on a solid support, more preferentially, by simple cross-linking.

According to the invention, the inactivation process may be used for treating a biological sample and notably a blood product or derivative of blood.

### SHORT DESCRIPTION OF THE FIGURES

FIG. 1 is a schematic illustration of the oxidation/reduction reaction of methionines.

### DETAILED DESCRIPTION OF THE INVENTION

### Definitions

Within the context of the invention, a “pathological prion protein” is meant to be the abnormal form of the prion protein (PrP). The prion protein PrP is generally a sialoglycoprotein anchored to the plasma membrane through a phosphatidyl glycolipid (GPI), present in the natural state in cells and involved in their normal operation. The normal form of the protein, i.e. non-pathological, is generally called PrPc. The “pathological” form of the PrP, called PrPsc, consists in an isoform of the non-pathological protein (including its intermediate or nucleic forms).

This pathological form of PrP is notably found in subjects affected with Creutzfeldt-Jakob disease (CJD). In a characteristic way, the occurrence of clinical signs and notably histological lesions, is preceded by a long clinically silent incubation period, during which the pathological form of the prion protein PrPsc accumulates in the central nervous system. Neither any modification of expression of the gene coding for PrP, nor any alteration of its translation in the affected subjects have been demonstrated. Two forms of CJD have been identified to this day. The sCJD form corresponds to the spontaneous form, occurring naturally in elderly subjects (generally around 65 years old). It is similar to a disease of ageing, without any identified infectious cause. The form vCJD corresponds to the expression in humans of the mad cow disease, due to the ingestion of beef contaminated with ESB. It affects a very small number of subjects (228 listed cases worldwide), relatively young subjects (20-30 years old). This form of prion is considered as transmissible through the blood.

By “inactivation” is meant that the prion protein loses its capability of inducing a change in the conformation of the native prion proteins and/or of inducing a neurodegenerative disease. Inactivation is not necessarily associated with degradation of the protein which, itself, implies degradation of the peptide structure. The inactivation of PrPsc may notably consist in a return to the non-pathological native form of the prion protein.

By “decontamination” of a sample or material, is meant that the pathological prion protein which may be present in/on said sample or material is inactivated. Preferentially, it is considered that a sample or material is decontaminated when it is no longer possible to measure any infectiosity associated with the prion protein PrPsc in said sample or material.

By “sample”, is meant any material source which may be contaminated with and by containing a pathological prion protein. Such a material source may for example be a biological sample, a cosmetic or pharmaceutical product, a product from genetic engineering, a food product, a beverage, this list not being limiting. Preferably, this is a biological sample, for example a biological liquid or a tissue or tissue extract, such as a spinal column tissue and notably spinal marrow. The sample may also be a composition derived from a human or animal source, such as growth hormones or cell extracts, such as pituitary extracts. Such a composition may actually be contaminated by a pathological prion protein. In the case of a biological liquid, the latter may be blood or a derivative, blood plasma, lymph, urine, milk, etc, this list not being limiting. Preferentially, the sample is a blood product or a derivative, for example a plasma derivative or a concentrate of plasma protein.

### Methionine Sulfoxide Reductase

MSRs are enzymatic proteins present in a large number of both eukaryotic and prokaryotic organisms, involved in the regeneration of proteins bearing methionine sulfoxide (MetSO) residues. More specifically, the MSRs give the possibility of catalyzing the reaction for reducing MetSOs into methionines. The oxidation of methionines within a protein, notably under the action of reactive oxygen derivatives (ROS), is often accompanied by a loss of functionality of said protein. It has been demonstrated that the prion protein, in its pathological form, contains a significant amount of MetSO (Canello et al. Biochemistry 2008, 47, 8866-8873).

The invention proposes the use of an MSR in the treatment of a sample or material which may be contaminated by a pathological prion protein, in order to inactivate said pathological prion protein.

According to the invention, the MSR used may be of natural origin, and notably of plant, bacterial or animal origin, or synthetic or semi-synthetic.

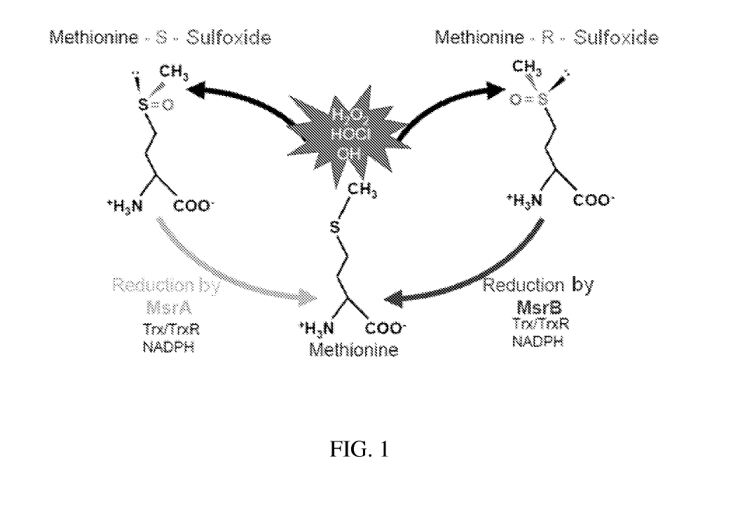

In humans notably, there exist two main forms of MSR, MSRA and MSRB, respectively involved in the reduction of methionine—S-sulfoxide and of methionine—R-sulfoxide (cf. FIG. 1). More specifically, a same gene msra (located on the chromosome 8p23.1) codes for four isoforms MSRA1 (RefSeq RNA NM_012331), MSRA2 (RefSeq RNA NM_001135670), MSRA3 (RefSeq RNA NM_001135671) and MSRA4 (RefSeq RNA NM_001199729), present in mitochondria, cytosol and/or cell nuclei. Also, there exists three genes msrb in humans, msrb1 (on the chromosome 16p13.3), msrb2 (on the chromosome 10p12) and msrb3 (on the chromosome 12q14.3), respectively coding for the isoforms MSRB1 (Ref. Seq RNA NM_016332), MSRB2 (Ref. Seq RNA NM_012228), MSRB3.A (Ref. Seq RNA NM_198080) and MSRB3.B (coded by three different variants: Ref. Seq RNA NM_001031679, Ref. Seq RNA NM_001193460, RefSeq RNA NM_001193461), present in mitochondria, endoplasmic reticulum, cytosol and/or cell nuclei.

The MSR may be a wild or native MSR, or a recombinant, derived or mutant MSR, having a sequence substantially homologous to native MSR. The expression “sequence substantially homologous” comprises any sequence subject to one or several substitutions, additions and/or deletions, preferably conservative operations. The expressions “conservative substitutions, additions and/or deletions” express any replacement, addition or suppression of an amino acid residue with another one, without any major alteration of the general conformation and/or of the biological activity (of reduction of the MetSOs into methionines) of MSR. Conservative substitution includes, without being limited thereto, the replacement with an amino acid having similar properties (such as for example the form, the polarity, the hydrogen bond potential, the acidity, basicity, hydrophobicity and other properties). Amino acids having similar properties are well known in the art.

The MSR used may be a variant of a wild MSR having an equivalent or superior biological activity as compared with the activity of the wild form, these variants notably including variants from natural allelic variations and/or from isoforms of MSR naturally found in individuals of a same species, and any form or degree of glycosylation or any other post-translation modification. Homologs or derivatives of MSR are also included, which have the same or a superior biological activity as compared with the activity of a wild form and/or which have a sequence identity of at least 80%, preferably at least 85%, still preferably at least 90%.

In a preferred embodiment, the MSR used is human MSR. Notably, the MSR may be any of the human MSRAs or MSRBs above. In another exemplary embodiment, the MSR used is a bacterial MSR.

It is also possible to use several MSRs simultaneously, which may, if required, be of different origins.

The MSR according to the invention may be prepared by all the standard purification techniques, by peptide synthesis and notably by chemical synthesis, by chemical engineering, or other technique.

In the case when the MSR is a recombinant MSR, it may be obtained with a standard process for producing recombinant proteins, comprising the transfer of a vector into a host cell, under conditions allowing expression of the recombinant protein coded by the vector, and the recovery of the thereby produced protein. The vector may be prepared according to methods currently used by the person skilled in the art, and the clones resulting from them may be introduced into the host cell, with standard methods, such as lipofection, electroporation or thermal shock. The host cell may notably be a bacterium, a yeast, a fungus or a mammal cell.

It is also possible to produce the MSR in a transgenic organism for example in a plant, or in the milk of a non-human transgenic mammal such as a goat, a rabbit or a pig. The secretion of MSR through the mammary glands, allowing its secretion into the milk of transgenic mammals, involves controlling the expression of the MSR in a tissue-dependent way. Such control methods are well known to the person skilled in the art. The control of the expression is carried out by means of sequences allowing expression of the protein towards a particular tissue of the animal. These are notably WAP, beta-casein, beta-lactoglobulin promoter sequences and peptide signal sequences. The process for extracting proteins of interest from the milk of transgenic animals is described in patent EP 0 264 166.

Any existing MSR isoform notably due to alternative splicing of the gene may be used. Also, it is possible to use as an MSR, a biologically active portion of a native MSR. By “biologically active portion”, is meant that the MSR used has biological activity (reduction of an MetSO into methionine) at least equivalent to the activity of the native protein.

According to the invention, for example, it is possible to use an MSRA of human origin, for example comprising a sequence such as:
the sequence SEQ ID No. 2 coding for MSRA1, which may be synthesized from the nucleotide sequence SEQ ID No. 1;
the sequence SEQ ID No. 4 coding for MSRA2, which may be synthesized from the nucleotide sequence SEQ ID No. 3
the sequence SEQ ID No. 6 coding for MSRA3, which may be synthesized from the nucleotide sequence SEQ ID No. 5
the sequence SEQ ID No. 8 coding for MSRA4, which may be synthesized from the nucleotide sequence SEQ ID No. 7.

In another example, an MSRB of human origin is used, for example comprising a sequence such as
the sequence SEQ ID No. 10 coding for MSRB1, which may be synthesized from the nucleotide sequence SEQ ID No. 9
the sequence SEQ ID No. 12 coding for MSRB2, which may be synthesized from the nucleotide sequence SEQ ID No. 11
the sequence SEQ ID No. 14 coding for MSRB3A, which may be synthesized from the nucleotide sequence SEQ ID No. 13
the sequence SEQ ID No. 16, SEQ ID No. 18 or SEQ ID No. 20, corresponding to three variants of MSRB3 which may be synthesized from the nucleotide sequence SEQ ID No. 15, SEQ ID No.17 or SEQ ID No. 19, respectively.

In an exemplary particular application, the MSR used comprises at least one biologically active portion of one of the isoforms of human MSRA selected from the peptide sequence SEQ ID No. 2, SEQ ID No. 4, SEQ ID No. 6 and SEQ ID No. 8.

In another exemplary particular application, the MSR used comprises at least one biologically active portion of one of the isoforms of human MSRB selected from the peptide sequence SEQ ID No. 10, SEQ ID No. 12, SEQ ID No. 14, SEQ ID No. 16, SEQ ID No. 18 and SEQ ID No. 20.

In a particular example, the MSR is a recombinant enzyme.

In another example, a combination is used of at least two MSRs each synthesized from one of the nucleotide sequences above and/or each having at least one biologically active portion of one of the peptide sequences above. Indeed, under certain conditions, a synergistic activity of MSRAs and MSRBs is observed when they are used simultaneously. In this case, the MSRs may be added in identical or different amounts, for example depending on the nature of the sample to be treated.

### Treatment of the Sample or of the Material

According to the invention, the sample or material to be treated is put into contact with at least one MSR for a sufficient time in order to allow inactivation of the potentially present pathological prion proteins. Preferentially, the sample is put into contact with the MSR for a period comprised between 10 and 120 minutes, and even more preferentially for a period comprised between 15 and 60 minutes.

Preferentially, the contacting is maintained until the infectious load has been reduced to below a titer measurable with techniques well known to the person skilled in the art, such as Western blot or infectiosity tests.

Advantageously, the contacting of the sample or material to be treated with the MSR is achieved at an alkaline pH, preferentially at a pH of more than 8 and still more preferentially of more than 9.

In an exemplary embodiment, the MSR is in the form of a solid or liquid composition, which may be directly added into the sample to be treated. In the case of a piece of equipment, such as a surgical instrument, said piece of equipment may be immersed in a solution comprising the MSR.

When the sample to be treated is a liquid sample, the step for contacting it with the MSR may be carried out with stirring, for example mechanical stirring, in order to optimize inactivation of the PrPscs.

Preferentially, the process according to the invention is conducted at a temperature of less than 100° C., more preferentially less than 80° C. and still more preferentially at a temperature of less than 60° C.

In certain cases, and notably for the treatment of a non-biological sample, it is possible to provide a heat treatment step, notably at temperatures above 60° C., preferentially above 80° C. and still more preferentially above 100° C. upstream and/or downstream from the step for inactivating PrPscs with MSR.

In certain cases, the use of MSR may be coupled with the use, either simultaneously or not, of a chemical compound which may denaturate the proteins, such as a detergent and notably sodium dodecyl sulfate (SDS), or a chaotropic salt and notably urea and guanidine salts.

Optionally, the inactivation process of the invention is applied in the presence of NADH or NADPH. In this particular embodiment, the process of the invention therefore further comprises the addition of NADH or NADPH, preferably to the sample which may be contaminated with a pathological prion protein, such as plasma or a plasma product. NADH or NADPH (hydrogenated dinucleotide adenine nicotinamide or hydrogenated phosphate dinucleotide adenine nicotinamide) is a coenzyme present in an intracellular way and is the main source of electrons used in biosynthetic reactions in the cell. It is also used in mechanisms for protection against oxidizing stress and reactive oxygen species.

In an embodiment, in order to amplify the inactivation, it is possible to regenerate the MSRs by adding an oxidoreductase, and more particularly thioredoxin (Trx) to the sample (Tarrago et al., The journal of biological chemistry Vol. 284, No. 28, p. 18963-18971, Jul. 10, 2009). Preferentially, the addition of Trx is accompanied by adding either simultaneously or sequentially NADPH. The oxidoreductase may be added to the sample to be treated at the same time as the MSR, or after a period of reaction of the MSR in the sample, or in certain cases before adding the MSR.

In an embodiment suitable for an industrial application, the MSR is immobilized according to diverse immobilization techniques known to the person skilled in the art. The MSR may then be in contact with the sample to be treated. These various immobilization techniques prevent migration of the MSR in the product and give the possibility of doing without certain purification steps. With these different techniques, the MSR may also be reused and provide cost-effectiveness of the applied process.

Typically, the MSR is immobilized by covalent bonding on a solid support activated beforehand in order to generate amide bonds with the primary amines available on the MSR. For amidation induced by cyanogen bromide, supports of the polysaccharide type such as cellulose, agarose and sepharose and supports in the form of a silica gel or porous glass are preferentially used. For amidation by activation with a carbodiimide, acid supports notably functionalized silicas or plastics are preferentially used. For amidation by activation with ethyl chloroformate, diol supports, notably polysaccharides, silica gel or porous glass, are preferentially used.

In particular, the MSR is immobilized by simple cross-linking by means of a cross-linking agent, notably glutaraldehyde.

### Applications

The process according to the invention may be used for treating any kind of sample or material, either solid or liquid, which may contain pathological prion proteins.

Notably, the process according to the invention may be applied to any liquid, food or biological product, piece of equipment, device, instrument etc.

For example, the MSRs according to the invention may be used in processes for sterilizing medical equipment, such as surgical instruments. Also, MSRs may be used for disinfecting surfaces and notably laboratory benches or the floor of certain plants/factories in which products which may be contaminated by PrPscs are treated.

MSRs may also be used during the preparation of pharmaceutical or cosmetic compositions and notably of compositions incorporating a blood-derived product.

Also, MSRs may be used in processes for preparing and/or conditioning food products, and notably food products incorporating meat products or products from animals, such as milk.

### Validation of the Inactivation Process by Titration of Infectiosity

The efficiency of inactivation of pathological prion proteins in a sample or material by the process according to the invention may be checked by titrating infectiosity, the marker of which is exactly said pathological conformation protein.

In the case of a sample, notably a biological sample, it is possible to proceed with several dilutions, most particularly with serial or successive dilutions of said sample before titration. These dilutions give the possibility of refining the quantification of infectiosity. For example, the treated sample is diluted in a load buffer according to a geometrical progression, also called “a dilution step”. The dilution step is preferably 3. Each dilution point is then subject to a test for detection of pathological prion proteins. For comparison purposes, it is possible to conduct the same titration in parallel on a non-treated sample.

Titration may be carried out by means of any titration method known to the person skilled in the art. In particular, it may be carried out on the model of the method called “TCIA” described in documents WO2005022148 and WO2006117483 (notably the reference examples A and B), or of the method combining “TCIA” and “PMCA” described in application WO2009/125139.

In an embodiment of the invention, the method for calculating the titer is the method of Spearman-Karber (Schmidt N. J., Emmous R. W., Diagnostic Procedures for viral, ricketsial and chlaveydial Infection, 1989, 6^{th} Edition). This method assumes dilution of the sample to be tested according to a geometrical progression, i.e. with a constant ratio between successive dilutions, and seeding of a constant volume (generally 0.150 ml) of each dilution in at least five wells. The most currently used dilution factor is the decimal factor. This method is described in detail in patent application WO2010/026346.

The process of the invention will be better understood by means of the additional description which follows and which does not limit the scope of the invention.

### EXAMPLE 1

### Test of Inactivation of the Pathogenic Prion by MSRA or MSRB2

Various MSR concentrations are incubated in a sample of minced brain containing a mixture of PrPc/PrPsc and the impact of MSR is measured by semi-quantitative dosage of resistance to proteinase K analyzed on a Western blot. More particularly, an amount of NADH or NADPH playing the role of a coenzyme may be added into the incubation medium.

### Material

The minced brain contains infectious forms of prion and the MSRA or MSRB2 used is in the form of cryotubes prepared from 2*100 μg of MSRA or MSRB2 (Abcam) purified to 95% at 1 mg/ml with a specific activity of 36U/mg, a unit leading to the oxidation of 1 μmol of NADPH at 30° C. at pH 7.4, i.e. 2*3.6 IU. The tubes provide aliquots per 0.5 IU and are therefore a total number of 14.

### Test with Proteinase K

The test with proteinase K is used, followed by development on a Western blot in order to detect PrPsc. Indeed, PrPsc is more resistant to treatments based on protease. Thus, the digestion by the proteinase is a preliminary step for Western blot and mainly digests PrPc. The subsequent detection step therefore no longer detects the non-pathological form of the protein since the latter has been digested by the protease.

### Method

i) The minced brain sample is diluted to the two lowest analyzable concentrations for the sensitivity test to proteinase K in a sodium phosphate buffer (disodium phosphate) 50 mM, pH=7.5, 50 mM of NaCl (dilution buffer);

ii) 0.5 IU or 1 IU (1 cryotube or 2 cryotubes) are added by rinsing the cryotube with 50 μl of dilution buffer in 1 ml of each concentration i.e. 2 enzyme concentrations per prion concentration. Each sample is made independently and in duplicate (8 samples all in all). One ml controls without any enzyme at the same dilutions with an addition of 60 μl of dilution buffer are also made (2 samples);

iii) The 10 tubes are incubated for 1 h at 37° C.; and

iv) The sensitivity test to proteinase K is conducted on each of the samples.

### EXAMPLE 2

### Test of Inactivation of the Pathogenic Prion by MSRA or MSRB2

In this example, various MSRA and/or MSRB2 concentrations are incubated in samples of minced human brains containing a mixture of PrPc/PrPsc and the effect of the MSRs is analyzed by measuring the delay in amplification of PrPsc by means of the RT-QuiC (<<real-time quaking induced conversion>>) technique (Ryuichiro Atarashi et al., Prion Volume 5 Issue 3, 150-153 July/August/September 2011). An amount of NADH or NADPH playing the role of a coenzyme is added in certain cases into the incubation medium.

### Material & Method

### Preparation of Microsome Fractions

Samples of human brains of patients deceased from the sporadic Creutzfeldt-Jakob disease (sCJD) or from the variant of the Creutzfeldt-Jakob disease (vCJD), available at the Institut du Cerveau et de la Moelle Epinière (ICM—Hôpital Pitié Salpêtrière, Paris, France), were used.

The samples were milled and taken up in PBS, and then clarified by means of a first centrifugation (350×g for 5 minutes at room temperature—i.e. about 25° C.).

The supernatants were collected, and then subject to a second centrifugation (10,000×g for 10 minutes at room temperature).

At the end of this second centrifugation, the supernatants were collected and subject to ultracentrifugation (100,000×g for 1 hour at 4° C.).

The pellets were taken up in 100 μl of PBS (vCJD samples) or 200 μl of PBS (sCJD samples).

### Positive Controls

Each experiment was completed with a positive control, made under the treatment conditions (buffer, environment, temperature, MSR—A concentration) identical with those of the relevant microsome fraction. The positive controls contain an oxidized protein (MS01, Oxford Biomedical Research) for which the methionine sulfoxide level was monitored by Western blot by means of a highly specific rabbit polyclonal antibody of sulfoxide methionines.

### Enzymes

Active recombinant human MSRA purified to 95% at 1 mg/ml with a specific activity of 36 U/mg, a unit leading to the oxidation of 1 μmol of NADPH at 30° C. at pH 7.4 (ab82728, Abcam®) was diluted to 1/10 and 1/100.

Active recombinant human MSRB2 purified to 90% at 1 mg/ml with a specific activity such that 1 nmol of enzyme allows reduction of 3 nmol of methionine sulfoxide bond in 1 minute at 37° C. (ab95916, Abcam®) was diluted to 1/10 and 1/100.

The enzymatic activity of both MSRs was validated by an activity test consisting of putting a reference protein (MSo1B, Oxford Biomedical Research) in contact with each of said enzymes, in the presence of NADPH. The analysis was made by Western blot by means of a highly specific rabbit polyclonal antibody of methionine sulfoxides. A significant reduction of the methionine sulfoxides was ascertained in the samples treated with either one of the MSRs comparatively with the control samples.

In all the experiments using NADPH (N9660, Sigma Aldrich), NADPH suspended in 0.01N NaOH was used so as to be at 500 μg/ml (i.e. 600 nmol/ml). Before use, the NADPH was diluted in PBS so as to be at 60 nmol/ml.

### Preparation of the Samples

The experiments were conducted with two different concentrations of microsome fractions vCDJ and sCDJ (dil −5 (10⁻⁵) and dil −2 (10⁻²)) for each of the enzyme concentrations (dilutions to 1/10 and 1/100), in order to act on the substrate/enzyme ratio, in the presence or in the absence of 5 μl of NADPH (+NADPH/−NADPH).

After treatment with MSRA or MSRB2 for 1 hour, the samples were again diluted to 1/100 for the preparations dil −5 (10⁻⁵) and to 1/1000 for the preparations dil −2 (10⁻²) for at most getting rid of a matrix effect while remaining in prion concentrations which may be titrated for the analysis step.

Two negative controls (5 μl MSRA 1/10+5 μl PBS+5 μl NADPH; 5 μl MSRB2 1/10+5 μl PBS+5 μl NADPH) were also analyzed.

Each experiment was conducted in triplicate.

### RT-Quic Technique

The analyses were carried out with the “RT-Quic” method as shown in Atarashi et al., 2011. To summarize, the method is based on an amplification of the conversion of the normal prion protein (PrPc) into an abnormal prion protein (PrPsc) in the samples. The fluorescence emission related to the presence of Thioflavine T (ThT) in the samples is then measured. ThT was incorporated to the newly formed amyloid fibrils in the samples during the conversion of PrPc into PrPsc. This method allows detection of all the forms of abnormal PrP and information to be provided in real time.

In the samples treated with the MSRs, a delay in the amplification with respect to the observed amplification time for the untreated controls is expected to be observed. In order to analyse the results, the TT (threshold time) notion was defined as being the time for which the value of the amplification curve exceeds a threshold arbitrarily set to 60,000 FU (fluorescence units), a value beyond the values observed for the negative controls.

For each condition (type of prion, dilution of the microsome fraction and pre-analytical dilution), a TT was defined from the results observed for the positive controls and was used as a reference. A value of TT for the treated samples greater than the maximum value of the TTs of the reference interval corresponds to a significantly smaller amount of PrPsc than the amount of PrPsc present in the control samples. This difference thus demonstrates a delay in the amplification of PrPsc, a result of the activity of the MSRs.

### Results

The amplification delay results for the analyzed samples are summarized in the tables below.

TABLE 1
Effect of MSRA or MSRB in the vCDJ samples

| | | | | | (TT) |
|---|---|---|---|---|---|
| | MSR | FM | | Analytical | in |
| MSR | dilution | dilution | NADPH | dilution | hours |
| MSRA | 1/10 | 10⁻⁵ | No | 1/100 | 36 |
| MSRB | 1/100 | 10⁻⁵ | No | 1/100 | 38 |
| MSRB | 1/10 | 10⁻⁵ | No | 1/100 | 40 |
| MSRA | 1/100 | 10⁻⁵ | No | 1/100 | 40 |
| MSRA | 1/10 | 10⁻⁵ | Yes | 1/100 | 47 |
| MSRB | 1/10 | 10⁻⁵ | Yes | 1/100 | 52 |
| MSRA | 1/100 | 10⁻⁵ | Yes | 1/100 | 54 |
| MSRB | 1/100 | 10⁻⁵ | Yes | 1/100 | 58 |
| Control (PBS) | | 10⁻⁵ | No | 1/100 | 33-35 |

TABLE 2
Comparison of the amplification delays in the presence or in the absence of
NADPH (MSRA and MSRB combined) in the vCDJ samples

| | TT | Control | MSRs |
|---|---|---|---|
| WITHOUT NADPH | hours | 33-35 | 36-40 |
| | minutes | 1980-2100 | 2130-2415 |
| WITH NADPH | hours | 33-35 | 47-58 |
| | minutes | 1980-2100 | 2790-3480 |

Thus, a systematic delay is observed in the vCDJ samples treated with the MSRs. The substantially more significant effect in the presence of NADPH was statistically validated by a “Student Test”.

TABLE 3
Effects of MSRA in sCDJ samples

| | | | | | (TT) |
|---|---|---|---|---|---|
| | MSR | FM | | Analytical | in |
| MSR | dilution | dilution | NADPH | dilution | hours |
| MSRA | 1/10 | 10⁻² | Yes | No | 35 |
| MSRA | 1/100 | 10⁻² | Yes | No | 38 |
| MSRA | 1/10 | 10⁻² | No | No | 42 |
| MSRA | 1/100 | 10⁻² | No | No | 46 |
| MSRA | 1/10 | 10⁻² | Yes | 1/1000 | 22 |
| MSRA | 1/100 | 10⁻² | Yes | 1/1000 | 28 |
| MSRA | 1/10 | 10⁻² | No | 1/1000 | 46 |
| MSRA | 1/100 | 10⁻² | No | 1/1000 | 68 |
| Control (PBS) | | 10⁻² | | No | 21-34.5 |
| Control (PBS) | | 10⁻² | | 1/1000 | 5-21 |

A systematic delay is also observed in the sCDJ samples treated with MSRA (35-46 versus 21-34.5 for the control in the samples without any analytical dilution; 22-68 versus 5-21 for the control in analytical dilution 1/1000—see table 4 below).

TABLE 4
Comparison of the TTs in the presence or in the absence of MSRA in
sCDJ samples

| | Control | MSRA |
|---|---|---|
| Delay | (without any analytical dilution) | (without any analytical dilution) |
| hours | 21-34.5 | 35-46 |
| minutes | 1260-2070 | 2100-2760 |
| | Control | MSRA |
| Delay | (with analytical dilution) | (with analytical dilution) |
| hours | 5-21 | 22-68 |
| minutes | 300-1260 | 1320-4080 |

A substantially more significant effect in the presence of NADPH was also observed (not reported in the tables).

A reduction in the activity for amplifying the abnormal prion protein is actually observed in the samples treated with exogenous MSRs. Further, the effect is improved in the presence of NADPH. Further, a synergistic effect of MSRAs and MSRB is expected to be obtained in the samples treated with these two enzymes.

1. A process for inactivating pathological prion proteins (PrPsc) in a sample or a material which may be contaminated by a pathological prion protein, wherein the sample or the material which may be contaminated by a pathological prion protein is put into contact with at least one methionine sulfoxide reductase (MSR).

2. The inactivation process according to claim 1, wherein the methionine sulfoxide reductase is a human MSR.

3. The inactivation process according to claim 1, wherein the methionine sulfoxide reductase is a methionine sulfoxide reductase A (MSRA).

4. The inactivation process according to claim 1, wherein the methionine sulfoxide reductase is a methionine sulfoxide reductase B (MSRB).

5. The inactivation process according to claim 1, wherein the methionine sulfoxide reductase is of natural origin.

6. The inactivation process according to claim 1, wherein the methionine sulfoxide reductase is of synthetic origin.

7. The inactivation process according to claim 1, wherein the methionine sulfoxide reductase was produced by genetic recombination.

8. The inactivation process according to claim 1, wherein the sample which may be contaminated by a pathological prion protein is a biological product.

9. The inactivation process according to claim 1, wherein the step for inactivating pathological prion proteins with MSR is conducted at a temperature below 60° C.

10. The inactivation process according to claim 1, further comprising a step for heat treatment of the sample or material which may be contaminated by a pathological prion protein, at a temperature above 60° C., before or after the step for inactivating the pathological prion proteins with MSR.

11. The inactivation process according to claim 1, comprising the addition of NADH or NADPH to the sample or material which may be contaminated by a pathological prion protein.

12. The inactivation process according to claim 1, wherein the MSR is immobilized on a solid support.

13. The inactivation process according to claim 8, wherein the sample which may be contaminated by a pathological prion protein is a blood product.

14. The inactivation process according to claim 13, wherein the sample which may be contaminated by a pathological prion protein is plasma or a plasma product.

15. The inactivation process according to claim 12, wherein the MSR is immobilized on the solid support by a covalent bond.

16. The inactivation process according to claim 15, wherein the MSR is immobilized on the solid support by simple cross-linking.


SEQUENCE LISTING (20 sequences)

SEQ ID NO: 1 - DNA, 1492 bp, Homo sapiens
  CDS (198)..(905)
cagccggtacggccccgggtttgggcaacctcgattacgggcggcctccagccccgccagcagcgccccgcgcccgcccgcccgcgcccctgccgccccccggttccggccgcggaccccactctctgccgttccggctgcggctccgctgccggtagcgccgtcccccgggaccacccttcggctggcgccctcccatgctctcggccacccggagggcttgccagctcctcctcctccacagcctctttcccgtcccgaggatgggcaactcggcctcgaacatcgtcagcccccaggaggccttgccgggccggaaggaacagacccctgtagcggccaaacatcatgtcaatggcaacagaacagtcgaacctttcccagagggaacacagatggctgtatttggaatgggatgtttctggggagctgaaaggaaattctgggtcttgaaaggagtgtattcaactcaagttggttttgcaggaggctatacttcaaatcctacttataaagaagtctgctcagaaaaaactggccatgcagaagtcgtccgagtggtgtaccagccagaacacatgagttttgaggaactgctcaaggtcttctgggagaatcacgacccgacccaaggtatgcgccaggggaacgaccatggcactcagtaccgctcggccatctacccgacctctgccaagcaaatggaggcagccctgagctccaaagagaactaccaaaaggttctttcagagcacggcttcggccccatcactaccgacatccgggagggacagactttctactatgcggaagactaccaccagcagtacctgagcaagaaccccaatggctactgcggccttgggggcaccggcgtgtcctgcccagtgggtattaaaaaataattgctccccacatggtgggcctttgaggttccagtaaaaatgctttcaacaaattgggcaatgcttgtgtgattcacaatcgtggcatttaaagtgcacaaagtacaaaggaatttatacagattgggtttaccgaagtataatctataggaggcgcgatggcaagttgataaaatgtgacttatctcctaataagttatggtgggagtggagctgtgcagtttcctgtgtcttctggggtctgagtgaagatagcagggatgctgtgttcacccttcttggtagaagctaaggtgtgagctgggaggttgctggacaggatgggggaccccagaagtcctttatctgtgctctctgcccgccagtgccttacaatttgcaaacgtgtatagcctcagtgactcattcgctgaaatccttcgctttaccaaatctagacatacataaggggctttctctcccttttcagccctctctgtgcagagaaaagatgtgagtccgcttgatgaattctaatgctttgcttagagctatgagaaatgtttgttttaataaaaacctacagtccaataatgccaaaaaaaaaa

SEQ ID NO: 2 - protein, 235 aa, Homo sapiens
MLSATRRACQLLLLHSLFPVPRMGNSASNIVSPQEALPGRKEQTPVAAKHHVNGNRTVEPFPEGTQMAVFGMGCFWGAERKFWVLKGVYSTQVGFAGGYTSNPTYKEVCSEKTGHAEVVRVVYQPEHMSFEELLKVFWENHDPTQGMRQGNDHGTQYRSAIYPTSAKQMEAALSSKENYQKVLSEHGFGPITTDIREGQTFYYAEDYHQQYLSKNPNGYCGLGGTGVSCPVGIKK

SEQ ID NO: 3 - DNA, 1372 bp, Homo sapiens
  CDS (198)..(785)
cagccggtacggccccgggtttgggcaacctcgattacgggcggcctccagccccgccagcagcgccccgcgcccgcccgcccgcgcccctgccgccccccggttccggccgcggaccccactctctgccgttccggctgcggctccgctgccggtagcgccgtcccccgggaccacccttcggctggcgccctcccatgctctcggccacccggagggcttgccagctcctcctcctccacagcctctttcccgtcccgaggatgggcaactcggcctcgaacatcgtcagcccccaggaggccttgccgggccggaaggaacagacccctgtagcggccaaacatcatgtcaatggcaacagaacagtcgaacctttcccagagggaacacagatggctgtatttgaaaaaactggccatgcagaagtcgtccgagtggtgtaccagccagaacacatgagttttgaggaactgctcaaggtcttctgggagaatcacgacccgacccaaggtatgcgccaggggaacgaccatggcactcagtaccgctcggccatctacccgacctctgccaagcaaatggaggcagccctgagctccaaagagaactaccaaaaggttctttcagagcacggcttcggccccatcactaccgacatccgggagggacagactttctactatgcggaagactaccaccagcagtacctgagcaagaaccccaatggctactgcggccttgggggcaccggcgtgtcctgcccagtgggtattaaaaaataattgctccccacatggtgggcctttgaggttccagtaaaaatgctttcaacaaattgggcaatgcttgtgtgattcacaatcgtggcatttaaagtgcacaaagtacaaaggaatttatacagattgggtttaccgaagtataatctataggaggcgcgatggcaagttgataaaatgtgacttatctcctaataagttatggtgggagtggagctgtgcagtttcctgtgtcttctggggtctgagtgaagatagcagggatgctgtgttcacccttcttggtagaagctaaggtgtgagctgggaggttgctggacaggatgggggaccccagaagtcctttatctgtgctctctgcccgccagtgccttacaatttgcaaacgtgtatagcctcagtgactcattcgctgaaatccttcgctttaccaaatctagacatacataaggggctttctctcccttttcagccctctctgtgcagagaaaagatgtgagtccgcttgatgaattctaatgctttgcttagagctatgagaaatgtttgttttaataaaaacctacagtccaataatgccaaaaaaaaaa

SEQ ID NO: 4 - protein, 195 aa, Homo sapiens
MLSATRRACQLLLLHSLFPVPRMGNSASNIVSPQEALPGRKEQTPVAAKHHVNGNRTVEPFPEGTQMAVFEKTGHAEVVRVVYQPEHMSFEELLKVFWENHDPTQGMRQGNDHGTQYRSAIYPTSAKQMEAALSSKENYQKVLSEHGFGPITTDIREGQTFYYAEDYHQQYLSKNPNGYCGLGGTGVSCPVGIKK

SEQ ID NO: 5 - DNA, 1641 bp, Homo sapiens
  CDS (476)..(1054)
gagagagagaaagagggagagagagaggcagagggagagctgaggaaagaaaaaaaggcaagacttggcacagctcagtcaaatcagcttcttttgtctgctttctcggcttgagcttcaggaaagaaaaccgtcctggggtacagaaaaactcagaactttttggttttcaaacttagaaggctttttaagtctcttgggctatttgaaagtgttggtacatacaatgacgtttagtcaccagtattaagggaaataaaagcctttttcaaaacgaagcttccatagtgtccatgcatttgggaaatactatatttgattttttgcatgtatgattataccatgagagacaggattaatatagaagatggcaaggcaaatttctaattagagggaatattaattttctacaaaataaagtttgttcatcaatacaaacctgctttcaaatcaaatcagaaagacatccttcggaatgtgttcagaacccaaacatcatgtcaatggcaacagaacagtcgaacctttcccagagggaacacagatggctgtatttggaatgggatgtttctggggagctgaaaggaaattctgggtcttgaaaggagtgtattcaactcaagttggttttgcaggaggctatacttcaaatcctacttataaagaagtctgctcagaaaaaactggccatgcagaagtcgtccgagtggtgtaccagccagaacacatgagttttgaggaactgctcaaggtcttctgggagaatcacgacccgacccaaggtatgcgccaggggaacgaccatggcactcagtaccgctcggccatctacccgacctctgccaagcaaatggaggcagccctgagctccaaagagaactaccaaaaggttctttcagagcacggcttcggccccatcactaccgacatccgggagggacagactttctactatgcggaagactaccaccagcagtacctgagcaagaaccccaatggctactgcggccttgggggcaccggcgtgtcctgcccagtgggtattaaaaaataattgctccccacatggtgggcctttgaggttccagtaaaaatgctttcaacaaattgggcaatgcttgtgtgattcacaatcgtggcatttaaagtgcacaaagtacaaaggaatttatacagattgggtttaccgaagtataatctataggaggcgcgatggcaagttgataaaatgtgacttatctcctaataagttatggtgggagtggagctgtgcagtttcctgtgtcttctggggtctgagtgaagatagcagggatgctgtgttcacccttcttggtagaagctaaggtgtgagctgggaggttgctggacaggatgggggaccccagaagtcctttatctgtgctctctgcccgccagtgccttacaatttgcaaacgtgtatagcctcagtgactcattcgctgaaatccttcgctttaccaaatctagacatacataaggggctttctctcccttttcagccctctctgtgcagagaaaagatgtgagtccgcttgatgaattctaatgctttgcttagagctatgagaaatgtttgttttaataaaaacctacagtccaataatgccaaaaaaaaaa

SEQ ID NO: 6 - protein, 192 aa, Homo sapiens
MCSEPKHHVNGNRTVEPFPEGTQMAVFGMGCFWGAERKFWVLKGVYSTQVGFAGGYTSNPTYKEVCSEKTGHAEVVRVVYQPEHMSFEELLKVFWENHDPTQGMRQGNDHGTQYRSAIYPTSAKQMEAALSSKENYQKVLSEHGFGPITTDIREGQTFYYAEDYHQQYLSKNPNGYCGLGGTGVSCPVGIKK

SEQ ID NO: 7 - DNA, 1716 bp, Homo sapiens
  CDS (620)..(1129)
gagagagagaaagagggagagagagaggcagagggagagctgaggaaagaaaaaaaggcaagacttggcacagctcagtcaaatcagcttcttttgtctgctttctcggcttgagcttcaggaaagaaaaccgtcctggggtacagaaaaactcagaactttttggttttcaaacttagaaggctttttaagtctcttgggctatttgaaagtgttggtacatacaatgacgtttagtcaccagtattaagggaaataaaagcctttttcaaaacgaagcttccatagtgtccatgcatttgggaaatactatatttgattttttgcatgtatgattataccatgagagacaggattaatatagaagatggcaaggcaaatttctaattagagggaatattaattttctacaaaataaagtttgttcatcaatacaaacctgctttcaaatcaaatcagaaagacatccttcggaatgtgttcagaaccaattagtgacaacactgaagaccagaaaggcaagctgaagacaccagacttcgcttgaagggcaaacaagaaatccaaacatcatgtcaatggcaacagaacagtcgaacctttcccagagggaacacagatggctgtatttggaatgggatgtttctggggagctgaaaggaaattctgggtcttgaaaggagtgtattcaactcaagttggttttgcaggaggctatacttcaaatcctacttataaagaagtctgctcagaaaaaactggccatgcagaagtcgtccgagtggtgtaccagccagaacacatgagttttgaggaactgctcaaggtcttctgggagaatcacgacccgacccaaggtatgcgccaggggaacgaccatggcactcagtaccgctcggccatctacccgacctctgccaagcaaatggaggcagccctgagctccaaagagaactaccaaaaggttctttcagagcacggcttcggccccatcactaccgacatccgggagggacagactttctactatgcggaagactaccaccagcagtacctgagcaagaaccccaatggctactgcggccttgggggcaccggcgtgtcctgcccagtgggtattaaaaaataattgctccccacatggtgggcctttgaggttccagtaaaaatgctttcaacaaattgggcaatgcttgtgtgattcacaatcgtggcatttaaagtgcacaaagtacaaaggaatttatacagattgggtttaccgaagtataatctataggaggcgcgatggcaagttgataaaatgtgacttatctcctaataagttatggtgggagtggagctgtgcagtttcctgtgtcttctggggtctgagtgaagatagcagggatgctgtgttcacccttcttggtagaagctaaggtgtgagctgggaggttgctggacaggatgggggaccccagaagtcctttatctgtgctctctgcccgccagtgccttacaatttgcaaacgtgtatagcctcagtgactcattcgctgaaatccttcgctttaccaaatctagacatacataaggggctttctctcccttttcagccctctctgtgcagagaaaagatgtgagtccgcttgatgaattctaatgctttgcttagagctatgagaaatgtttgttttaataaaaacctacagtccaataatgccaaaaaaaaaa

SEQ ID NO: 8 - protein, 169 aa, Homo sapiens
MAVFGMGCFWGAERKFWVLKGVYSTQVGFAGGYTSNPTYKEVCSEKTGHAEVVRVVYQPEHMSFEELLKVFWENHDPTQGMRQGNDHGTQYRSAIYPTSAKQMEAALSSKENYQKVLSEHGFGPITTDIREGQTFYYAEDYHQQYLSKNPNGYCGLGGTGVSCPVGIKK

SEQ ID NO: 9 - DNA, 1386 bp, Homo sapiens
  misc_feature (420)..(422): codon correspondant a un residu selenocysteine
ggaagccgggattcgccctccggggagcgattggtcctcgggaggggcggggaggtggacgcgggtaccggcggtcgtcgggtcggcagcctttggtcagttggcagcggcaagcgcgctgcggttccggtggcgccatgtcgttctgcagcttcttcgggggcgaggttttccagaatcactttgaacctggcgtttacgtgtgtgccaagtgtggctatgagctgttctccagccgctcgaagtatgcacactcgtctccatggccggcgttcaccgagaccattcacgccgacagcgtggccaagcgtccggagcacaatagatctgaagccttgaaggtgtcctgtggcaagtgtggcaatgggttgggccacgagttcctgaacgacggccccaagccggggcagtcccgattctgaatattcagcagctcgctgaagtttgtccctaaaggcaaagaaacttctgcctcccagggtcactaggcgggcagcccacacccaccccagacggccaccacactgaggccacacgttggccattccaccttggagttggaaccctgggcgtcgagacaggaaggcagggcgcagtggttgaaacatcaggacactcccaaggccccggctctgaacaagaccttttcgtttcttggaaaagagactcatttgctgatggttcatgccttctgctgggacaggcctgggctgtgcagccacactgtcggctgacttagccccctgctcactctaggtgcctccaggaggtgagccctgggtgcagctggtctctgaatgacgttacaccctcaccttcttttcctggccctgtctctggactctcccctgtgaggcccaattccaagacagactctcgtcctcaccgaagcttaggcccacatctcccaggctgcttaggagacagaatggaaacggaggccgcccctgccagccgccctggccctggtcactgcatgatccgctctggtcaaacccttccaggccagccagagtggggatggtctgtgacctgctgggaaggcaggctgatggggcacacccttggcctctcgtccacgaggggagaaacctaaaccctgtttcacaatctgtgcggaagtagcttgcctcacttctgcttaggaaagcggctgttgctccataactctaaccagcacagggctgaggcctgcagtgcacacctgcagggaggcccttcccaaggtgtggtgactgtgccttactgtacatgctcggaggcctggccatataggagggtgggtgatgctgaaatcaccccccatcttaagtaattactttctggagtaatcaggtggaaatccatagacaaatgaaacattcagaaaaaaaaaaaaaaaaaaaa

SEQ ID NO: 10 - protein, 116 aa, Homo sapiens
  MISC_FEATURE (95)..(95): Xaa correspond au residu selenocysteine
MSFCSFFGGEVFQNHFEPGVYVCAKCGYELFSSRSKYAHSSPWPAFTETIHADSVAKRPEHNRSEALKVSCGKCGNGLGHEFLNDGPKPGQSRFXIFSSSLKFVPKGKETSASQGH

SEQ ID NO: 11 - DNA, 1811 bp, Homo sapiens
  CDS (112)..(660)
agggaggccgactagcgcaggcggacgggagagaatgggggaggggtgggatgggagcaggggcagagacgggcagagggcagagggcggagcggcgccggagcgggcgtcatggcgcggctcctctggttgctccggggcctgaccctcggaactgcgcctcggcgggcggtgcggggccaagcgggcggcggcgggcccggcaccgggccgggactgggggaggcagggtctcttgcaacgtgtgagctgcctcttgccaagagtgagtggcaaaagaaactaaccccggagcagttctacgtcacaagagaaaagggaacggaaccgcctttcagtgggatctacctgaataacaaggaagcaggaatgtatcattgcgtgtgctgcgacagtccactcttcagttctgagaaaaagtactgctctggcactgggtggccttcgttttccgaggctcatggtacgtctggctctgatgaaagccacacagggatcctgagacgtctggatacctcgttaggatcagctcgcacagaggttgtctgcaagcagtgtgaagctcatctaggtcacgtgtttcctgatggacctgggcccaatggtcagaggttttgcatcaacagtgtggctttgaagttcaaaccaaggaaacactgaccatcttcaagagtcccgttcccttgccaccccttcacgtgcaccctcaatttccacaattcacttgaatgacttgttttatttgcaataaaactgggctgaatttgctgctgtctccagcgagtcattgcttctcttaatttatttacctggaatcaacttaatcctgtgtgttaggctgttcttgtgttgctataaagaagtacctgatcaggatctgggagaatttgaaaaaaaaagaaaaactagaaaaataaacaaaattaaaaagaaaaaaaaatacctgagactgagtaacttataaagaaaagaggtttaattgactcacagttctgcagggtgtacagaaagcatggtgccagcatttgctcggattctggggagacctgagggagcttttactcatggcagaaggtgaagggggagccggcgtctcacatggcaaagcaggagcaagagagagtggtagggaggtgccacatacttttaaacagccagatctctcaagaactcactcactgtcaccaggacagcaccaagccatgagggatccaccctcatgacccagttacctcccggcagggcccacctccggcactaggaattatacttcaacataagatttggagagggaaaacatccaaaccatatcacccaactcccacttggagtagaacttcacaaaacgacagagggacagctgtttccatcaaacacttccacagcttcacctgagacgagggcatgactctgggaacatcaggaggctgatggactattaagccaaacaattcctacaaaaattccctgcaccttccactgcgtttcagtccttgacagacataagtaagggatcaagtgagaagaggaaggaaaatacaggatccaaggaatcaacaatgctagcactgaagagcaggattacggaggagctgtgctctgggagcatatatggtgatcaatcagatgcatgacttttttttcaaaattttaaatgtcttgagcatgctacttccaaccattctacccacaaataaactgcaataggacttggttagatttcaatgattatgaataaatgttcaccttcataccttctggttacaaagctaaactttaaaatgtccaaataaaatattcactaaaattta

SEQ ID NO: 12 - protein, 182 aa, Homo sapiens
MARLLWLLRGLTLGTAPRRAVRGQAGGGGPGTGPGLGEAGSLATCELPLAKSEWQKKLTPEQFYVTREKGTEPPFSGIYLNNKEAGMYHCVCCDSPLFSSEKKYCSGTGWPSFSEAHGTSGSDESHTGILRRLDTSLGSARTEVVCKQCEAHLGHVFPDGPGPNGQRFCINSVALKFKPRKH

SEQ ID NO: 13 - DNA, 4307 bp, Homo sapiens
  CDS (127)..(705)
atatttggactcggctgcccgtgcccaggaatttcccgtcatgcctcccgccgccccgtccgtcgcccggagccggggagggagggagcgaggttcggacaccggcggcggctgcctggcctttccatgagcccgcggcggaccctcccgcgccccctctcgctctgcctctccctctgcctctgcctctgcctggccgcggctctgggaagtgcgcagtccgggtcgtgtagggataaaaagaactgtaaggtggtcttttcccagcaggaactgaggaagcggctaacacccctgcagtaccatgtcactcaggagaaagggaccgaaagtgcctttgaaggagaatacacacatcacaaagatcctggaatatataaatgtgttgtttgtggaactccattgtttaagtcagaaaccaaatttgactccggttcaggttggccttcattccacgatgtgatcaattctgaggcaatcacattcacagatgacttttcctatgggatgcacagggtggaaacaagctgctctcagtgtggtgctcaccttgggcacatttttgatgatgggcctcgtccaactgggaaaagatactgcataaattcggctgccttgtcttttacacctgcggatagcagtggcaccgccgagggaggcagtggggtcgccagcccggcccaggcagacaaagcggagctctagagtaatggagagtgatggaaacaaagtgtacttaatgcacagcttattaaaaaaatcaaaattgttatcttaatagatatattttttcaaaaactataagggcagttttgtgctattgatattttttcttcttttgcttaaacagaagccctggccatccatgtattttgcaattgactagatcaagaactgtttatagctttagcaaatggagacagctttgtgaaacttcttcacaagccacttataccctttggcattcttttctttgagcacatggcttcttttgcagtttttccccctttgattcagaagcagagggttcatggtcttcaaacatgaaaatagagatctcctctgcagtgtagagaccagagctgggcagtgcagggcatggagacctgcaagacacatggccttgaggcctttgcacagacccacctaagataaggttggagtgatgttttaatgagactgttcagctttgtggaaagtttgagctaaggtcatttttttttttctcactgaaagggtgtgaaggtctaaagtctttccttatgttaaattgttgccagatccaaaggggcatactgagtgttgtggcagagaagtaaacattaccacactgttaggcctttattttattttattttccatcgaaagcattggaggcccagtgcaatggctcacgcctgtgatcccagcactttgggaggccaaggcgggtggatcacgaggtcaggagatggagaccatcctggctaacatggtgaaaccccgtctctactaaaaatacgaaaaattagccaggcgtggtggtgggcacctgtagtcccagctactcaggaggctgaggcaggagaatggcgtgaacccggaaggcggagcttgcagttagccgagatcatgccactgcactccagcctacatgacaatgtgacactccatctcaaaaaataataataataacaatataagaactagctgggcatggtggcgcatgcatgtagtcccagctactcctgaggctcagtcaggagaatcgcttgaacttgggaggcggaggttgcagtgagctgagctcataccactgcactccagcctgaacagagtgagatcctgtcaaaaaagaaaagaaaaagaaagcagcattcaaatgtaagacaactgtaaaatattgagccccacttggtctaaaattcaaaaagaagaacgcctgtccatcgcctttttataagtccttctctccacacctaaaagcagctgcagctggaagggcacaaattccactgtgtaaaataaaatattaggggcaacacacttcatcaaggcagcaggaatgagagagagcagagaagatcaaggatgaagtcttgggtactgaaaaattcagtgctgggcagaaaaactgacagggcagtacaagtaacaaacagaatccaagtggggtggcccttgtgcacagagctccaggtgacctctggagagacatgggcattcacatggaaagctaaaacggaagctcaagtttcatactcaacataatcttctgtgtgacaaaggacaagccatgtagcctctctgtgcctatttcttcatgcataaactgggactcataatatttgtaaaatgtattgatactctcagggcaaattcactatattgctatacagttgagatcagtgttgtaaaattaaactgatctggttctaattgcctcaaaggccaaagcccaggcatttgaaatggaaagaagcagagaggaggctgacttagctgattggtatggaaacagttgggccaagagccagaatttccctttgtagcaacacggctagttttactttgagaagctctgctcagctgctttataacattaagtctggcggaatggatgtcactgtgcacaataaagttttcacaagtataaacaatggtgatgtaagtcaacattgctgtagccaggtgtgaaggttgtatggtgtgtgacgaatgtacatcatgtttgtaggtttggatgctaatcttgaattgtagtttaaaaaatacgtatttttgtaactctttgaaagtttatgaagactgacagctttccttgtaagcactaagagaaaaaaaagaaagagggacatttgacaattttaaagaaacaacaagaaattagaatgaaaatctgtgacaaacagcgtcagtgtggccatgtccacattcctacatgtctctctctacaagcacctctctaagaagcctgacatcccggtggactctttatagtcatgtacacttgattccagatgagctctggtcttatctggatgctcagataagaggtttctatctgagcatccagatgttccctcaggttccaagacatttcaccccaggccctgggttcactctggaattcgtaggcttcacgtctctctagaaatgacgtgtaaaatttaagaccagacctcagccatcagcgtccagaccatcctagaagtctttcccaatctcacagagaaagccctagtatttcccagtgaccccaggattccacgttggggtggccaaagaaataggtctctcagggctttgccacagcctccagcccatccttcagaggcacacacagcacctctcggctgctccagctctgtaggatagcctcccctggggtccgtgggacgcgggccacagtgttgaggtagacaaggaggatcagtgagaggcctcttccctctccacagagactggattgtcattgttccttcatttatatcgtagggcttaacatttcactcaaaaaaaagcccctctttttctaatccttagtctttgtttcaaggaaagccagtttttcttctaccacattttccaggatcgactttaagaaaaatgcaacatctattgaaaaaaagtggggtgtatgcatgtggtttaattccagattgcttttgggtttaagtggtatcaaatttcagtatatttctgtcttatgtgaaagaaatatattactaaaacgtcagtgagcaataatgtcagctgtcaagcactagatttatttttgcaggatatggagtgcaatgaactgagtcaatatggcaaggtgtatgtgatctgtgggagttatgccatttaacataggaagtgcatgggactttccctctctgcactccagctcttactgtaccattagaagatgcagaattctgttggtgtgcaaaaagtatagccttacattcaagcagaatggatctgaagaaagcagcaatatctgttactagagaacattcccatgtgtttaaactcttcacttcttagatgcatttaaattcttaatgcaaatgacgtagcaatttgaaaacttctccgtattacttgtgtttaaaatgtcttgctttaaatacaaaacaaatggtaaaggggattatcttttgtttagatggttaaatattatttttgccttagatagctttgtaataatttttctccagacagttcaacacttttgaaaaatgacatgaattttcattaaaaacccttttcctatgtttattgtatacaagaattatgcaataaaatttctttataaaaataaaaaaaaaaaaaaaaaa

SEQ ID NO: 14 - protein, 192 aa, Homo sapiens
MSPRRTLPRPLSLCLSLCLCLCLAAALGSAQSGSCRDKKNCKVVFSQQELRKRLTPLQYHVTQEKGTESAFEGEYTHHKDPGIYKCVVCGTPLFKSETKFDSGSGWPSFHDVINSEAITFTDDFSYGMHRVETSCSQCGAHLGHIFDDGPRPTGKRYCINSAALSFTPADSSGTAEGGSGVASPAQADKAEL

SEQ ID NO: 15 - DNA, 4434 bp, Homo sapiens
  CDS (275)..(832)
atatttggactcggctgcccgtgcccaggaatttcccgtcatgcctcccgccgccccgtccgtcgcccggagccggggagggagggagcgaggttcggacaccggcggcggctgcctggcctttccatgagcccgcggcggaccctcccgcgccccctctcgctctgcctctccctctgcctctgcctctgcctggccgcggctctgggaagtgcgcagtccgctcttgcccctgttctttgcttctcgttttgttggtgaagatatcacagtgatgtctgcattcaacctgctgcatttggtgacaaagagccagccagtagcccttcgagcctgtgggcttccctcagggtcgtgtagggataaaaagaactgtaaggtggtcttttcccagcaggaactgaggaagcggctaacacccctgcagtaccatgtcactcaggagaaagggaccgaaagtgcctttgaaggagaatacacacatcacaaagatcctggaatatataaatgtgttgtttgtggaactccattgtttaagtcagaaaccaaatttgactccggttcaggttggccttcattccacgatgtgatcaattctgaggcaatcacattcacagatgacttttcctatgggatgcacagggtggaaacaagctgctctcagtgtggtgctcaccttgggcacatttttgatgatgggcctcgtccaactgggaaaagatactgcataaattcggctgccttgtcttttacacctgcggatagcagtggcaccgccgagggaggcagtggggtcgccagcccggcccaggcagacaaagcggagctctagagtaatggagagtgatggaaacaaagtgtacttaatgcacagcttattaaaaaaatcaaaattgttatcttaatagatatattttttcaaaaactataagggcagttttgtgctattgatattttttcttcttttgcttaaacagaagccctggccatccatgtattttgcaattgactagatcaagaactgtttatagctttagcaaatggagacagctttgtgaaacttcttcacaagccacttataccctttggcattcttttctttgagcacatggcttcttttgcagtttttccccctttgattcagaagcagagggttcatggtcttcaaacatgaaaatagagatctcctctgcagtgtagagaccagagctgggcagtgcagggcatggagacctgcaagacacatggccttgaggcctttgcacagacccacctaagataaggttggagtgatgttttaatgagactgttcagctttgtggaaagtttgagctaaggtcatttttttttttctcactgaaagggtgtgaaggtctaaagtctttccttatgttaaattgttgccagatccaaaggggcatactgagtgttgtggcagagaagtaaacattaccacactgttaggcctttattttattttattttccatcgaaagcattggaggcccagtgcaatggctcacgcctgtgatcccagcactttgggaggccaaggcgggtggatcacgaggtcaggagatggagaccatcctggctaacatggtgaaaccccgtctctactaaaaatacgaaaaattagccaggcgtggtggtgggcacctgtagtcccagctactcaggaggctgaggcaggagaatggcgtgaacccggaaggcggagcttgcagttagccgagatcatgccactgcactccagcctacatgacaatgtgacactccatctcaaaaaataataataataacaatataagaactagctgggcatggtggcgcatgcatgtagtcccagctactcctgaggctcagtcaggagaatcgcttgaacttgggaggcggaggttgcagtgagctgagctcataccactgcactccagcctgaacagagtgagatcctgtcaaaaaagaaaagaaaaagaaagcagcattcaaatgtaagacaactgtaaaatattgagccccacttggtctaaaattcaaaaagaagaacgcctgtccatcgcctttttataagtccttctctccacacctaaaagcagctgcagctggaagggcacaaattccactgtgtaaaataaaatattaggggcaacacacttcatcaaggcagcaggaatgagagagagcagagaagatcaaggatgaagtcttgggtactgaaaaattcagtgctgggcagaaaaactgacagggcagtacaagtaacaaacagaatccaagtggggtggcccttgtgcacagagctccaggtgacctctggagagacatgggcattcacatggaaagctaaaacggaagctcaagtttcatactcaacataatcttctgtgtgacaaaggacaagccatgtagcctctctgtgcctatttcttcatgcataaactgggactcataatatttgtaaaatgtattgatactctcagggcaaattcactatattgctatacagttgagatcagtgttgtaaaattaaactgatctggttctaattgcctcaaaggccaaagcccaggcatttgaaatggaaagaagcagagaggaggctgacttagctgattggtatggaaacagttgggccaagagccagaatttccctttgtagcaacacggctagttttactttgagaagctctgctcagctgctttataacattaagtctggcggaatggatgtcactgtgcacaataaagttttcacaagtataaacaatggtgatgtaagtcaacattgctgtagccaggtgtgaaggttgtatggtgtgtgacgaatgtacatcatgtttgtaggtttggatgctaatcttgaattgtagtttaaaaaatacgtatttttgtaactctttgaaagtttatgaagactgacagctttccttgtaagcactaagagaaaaaaaagaaagagggacatttgacaattttaaagaaacaacaagaaattagaatgaaaatctgtgacaaacagcgtcagtgtggccatgtccacattcctacatgtctctctctacaagcacctctctaagaagcctgacatcccggtggactctttatagtcatgtacacttgattccagatgagctctggtcttatctggatgctcagataagaggtttctatctgagcatccagatgttccctcaggttccaagacatttcaccccaggccctgggttcactctggaattcgtaggcttcacgtctctctagaaatgacgtgtaaaatttaagaccagacctcagccatcagcgtccagaccatcctagaagtctttcccaatctcacagagaaagccctagtatttcccagtgaccccaggattccacgttggggtggccaaagaaataggtctctcagggctttgccacagcctccagcccatccttcagaggcacacacagcacctctcggctgctccagctctgtaggatagcctcccctggggtccgtgggacgcgggccacagtgttgaggtagacaaggaggatcagtgagaggcctcttccctctccacagagactggattgtcattgttccttcatttatatcgtagggcttaacatttcactcaaaaaaaagcccctctttttctaatccttagtctttgtttcaaggaaagccagtttttcttctaccacattttccaggatcgactttaagaaaaatgcaacatctattgaaaaaaagtggggtgtatgcatgtggtttaattccagattgcttttgggtttaagtggtatcaaatttcagtatatttctgtcttatgtgaaagaaatatattactaaaacgtcagtgagcaataatgtcagctgtcaagcactagatttatttttgcaggatatggagtgcaatgaactgagtcaatatggcaaggtgtatgtgatctgtgggagttatgccatttaacataggaagtgcatgggactttccctctctgcactccagctcttactgtaccattagaagatgcagaattctgttggtgtgcaaaaagtatagccttacattcaagcagaatggatctgaagaaagcagcaatatctgttactagagaacattcccatgtgtttaaactcttcacttcttagatgcatttaaattcttaatgcaaatgacgtagcaatttgaaaacttctccgtattacttgtgtttaaaatgtcttgctttaaatacaaaacaaatggtaaaggggattatcttttgtttagatggttaaatattatttttgccttagatagctttgtaataatttttctccagacagttcaacacttttgaaaaatgacatgaattttcattaaaaacccttttcctatgtttattgtatacaagaattatgcaataaaatttctttataaaaataaaaaaaaaaaaaaaaaa

SEQ ID NO: 16 - protein, 185 aa, Homo sapiens
MSAFNLLHLVTKSQPVALRACGLPSGSCRDKKNCKVVFSQQELRKRLTPLQYHVTQEKGTESAFEGEYTHHKDPGIYKCVVCGTPLFKSETKFDSGSGWPSFHDVINSEAITFTDDFSYGMHRVETSCSQCGAHLGHIFDDGPRPTGKRYCINSAALSFTPADSSGTAEGGSGVASPAQADKAEL

SEQ ID NO: 17 - DNA, 4598 bp, Homo sapiens
  CDS (439)..(996)
atatttggactcggctgcccgtgcccaggaatttcccgtcatgcctcccgccgccccgtccgtcgcccggagccggggagggagggagcgaggttcggacaccggcggcggctgcctggcctttccatgagcccgcggcggaccctcccgcgccccctctcgctctgcctctccctctgcctctgcctctgcctggccgcggctctgggaagtgcgcagtccgagtgacatcactgcctctcttcctgtgcgctggctttgacataagccagatggccaccgtggttggtaggcgcccaggctgcctggtacaggagttgatgaaacagaataggaagacgttttatggtcagctgtggaagcacagtgagactgcagctttgctaactcttgcccctgttctttgcttctcgttttgttggtgaagatatcacagtgatgtctgcattcaacctgctgcatttggtgacaaagagccagccagtagcccttcgagcctgtgggcttccctcagggtcgtgtagggataaaaagaactgtaaggtggtcttttcccagcaggaactgaggaagcggctaacacccctgcagtaccatgtcactcaggagaaagggaccgaaagtgcctttgaaggagaatacacacatcacaaagatcctggaatatataaatgtgttgtttgtggaactccattgtttaagtcagaaaccaaatttgactccggttcaggttggccttcattccacgatgtgatcaattctgaggcaatcacattcacagatgacttttcctatgggatgcacagggtggaaacaagctgctctcagtgtggtgctcaccttgggcacatttttgatgatgggcctcgtccaactgggaaaagatactgcataaattcggctgccttgtcttttacacctgcggatagcagtggcaccgccgagggaggcagtggggtcgccagcccggcccaggcagacaaagcggagctctagagtaatggagagtgatggaaacaaagtgtacttaatgcacagcttattaaaaaaatcaaaattgttatcttaatagatatattttttcaaaaactataagggcagttttgtgctattgatattttttcttcttttgcttaaacagaagccctggccatccatgtattttgcaattgactagatcaagaactgtttatagctttagcaaatggagacagctttgtgaaacttcttcacaagccacttataccctttggcattcttttctttgagcacatggcttcttttgcagtttttccccctttgattcagaagcagagggttcatggtcttcaaacatgaaaatagagatctcctctgcagtgtagagaccagagctgggcagtgcagggcatggagacctgcaagacacatggccttgaggcctttgcacagacccacctaagataaggttggagtgatgttttaatgagactgttcagctttgtggaaagtttgagctaaggtcatttttttttttctcactgaaagggtgtgaaggtctaaagtctttccttatgttaaattgttgccagatccaaaggggcatactgagtgttgtggcagagaagtaaacattaccacactgttaggcctttattttattttattttccatcgaaagcattggaggcccagtgcaatggctcacgcctgtgatcccagcactttgggaggccaaggcgggtggatcacgaggtcaggagatggagaccatcctggctaacatggtgaaaccccgtctctactaaaaatacgaaaaattagccaggcgtggtggtgggcacctgtagtcccagctactcaggaggctgaggcaggagaatggcgtgaacccggaaggcggagcttgcagttagccgagatcatgccactgcactccagcctacatgacaatgtgacactccatctcaaaaaataataataataacaatataagaactagctgggcatggtggcgcatgcatgtagtcccagctactcctgaggctcagtcaggagaatcgcttgaacttgggaggcggaggttgcagtgagctgagctcataccactgcactccagcctgaacagagtgagatcctgtcaaaaaagaaaagaaaaagaaagcagcattcaaatgtaagacaactgtaaaatattgagccccacttggtctaaaattcaaaaagaagaacgcctgtccatcgcctttttataagtccttctctccacacctaaaagcagctgcagctggaagggcacaaattccactgtgtaaaataaaatattaggggcaacacacttcatcaaggcagcaggaatgagagagagcagagaagatcaaggatgaagtcttgggtactgaaaaattcagtgctgggcagaaaaactgacagggcagtacaagtaacaaacagaatccaagtggggtggcccttgtgcacagagctccaggtgacctctggagagacatgggcattcacatggaaagctaaaacggaagctcaagtttcatactcaacataatcttctgtgtgacaaaggacaagccatgtagcctctctgtgcctatttcttcatgcataaactgggactcataatatttgtaaaatgtattgatactctcagggcaaattcactatattgctatacagttgagatcagtgttgtaaaattaaactgatctggttctaattgcctcaaaggccaaagcccaggcatttgaaatggaaagaagcagagaggaggctgacttagctgattggtatggaaacagttgggccaagagccagaatttccctttgtagcaacacggctagttttactttgagaagctctgctcagctgctttataacattaagtctggcggaatggatgtcactgtgcacaataaagttttcacaagtataaacaatggtgatgtaagtcaacattgctgtagccaggtgtgaaggttgtatggtgtgtgacgaatgtacatcatgtttgtaggtttggatgctaatcttgaattgtagtttaaaaaatacgtatttttgtaactctttgaaagtttatgaagactgacagctttccttgtaagcactaagagaaaaaaaagaaagagggacatttgacaattttaaagaaacaacaagaaattagaatgaaaatctgtgacaaacagcgtcagtgtggccatgtccacattcctacatgtctctctctacaagcacctctctaagaagcctgacatcccggtggactctttatagtcatgtacacttgattccagatgagctctggtcttatctggatgctcagataagaggtttctatctgagcatccagatgttccctcaggttccaagacatttcaccccaggccctgggttcactctggaattcgtaggcttcacgtctctctagaaatgacgtgtaaaatttaagaccagacctcagccatcagcgtccagaccatcctagaagtctttcccaatctcacagagaaagccctagtatttcccagtgaccccaggattccacgttggggtggccaaagaaataggtctctcagggctttgccacagcctccagcccatccttcagaggcacacacagcacctctcggctgctccagctctgtaggatagcctcccctggggtccgtgggacgcgggccacagtgttgaggtagacaaggaggatcagtgagaggcctcttccctctccacagagactggattgtcattgttccttcatttatatcgtagggcttaacatttcactcaaaaaaaagcccctctttttctaatccttagtctttgtttcaaggaaagccagtttttcttctaccacattttccaggatcgactttaagaaaaatgcaacatctattgaaaaaaagtggggtgtatgcatgtggtttaattccagattgcttttgggtttaagtggtatcaaatttcagtatatttctgtcttatgtgaaagaaatatattactaaaacgtcagtgagcaataatgtcagctgtcaagcactagatttatttttgcaggatatggagtgcaatgaactgagtcaatatggcaaggtgtatgtgatctgtgggagttatgccatttaacataggaagtgcatgggactttccctctctgcactccagctcttactgtaccattagaagatgcagaattctgttggtgtgcaaaaagtatagccttacattcaagcagaatggatctgaagaaagcagcaatatctgttactagagaacattcccatgtgtttaaactcttcacttcttagatgcatttaaattcttaatgcaaatgacgtagcaatttgaaaacttctccgtattacttgtgtttaaaatgtcttgctttaaatacaaaacaaatggtaaaggggattatcttttgtttagatggttaaatattatttttgccttagatagctttgtaataatttttctccagacagttcaacacttttgaaaaatgacatgaattttcattaaaaacccttttcctatgtttattgtatacaagaattatgcaataaaatttctttataaaaataaaaaaaaaaaaaaaaaa

SEQ ID NO: 18 - protein, 185 aa, Homo sapiens; identical to SEQ ID NO: 16

SEQ ID NO: 19 - DNA, 4296 bp, Homo sapiens
  CDS (137)..(694)
ctgcggggacagcccctgcctcagccgagaaggggagcagaagggttgcgccccgcgccagcggtgaggggccgaacggaaggagctcttgcccctgttctttgcttctcgttttgttggtgaagatatcacagtgatgtctgcattcaacctgctgcatttggtgacaaagagccagccagtagcccttcgagcctgtgggcttccctcagggtcgtgtagggataaaaagaactgtaaggtggtcttttcccagcaggaactgaggaagcggctaacacccctgcagtaccatgtcactcaggagaaagggaccgaaagtgcctttgaaggagaatacacacatcacaaagatcctggaatatataaatgtgttgtttgtggaactccattgtttaagtcagaaaccaaatttgactccggttcaggttggccttcattccacgatgtgatcaattctgaggcaatcacattcacagatgacttttcctatgggatgcacagggtggaaacaagctgctctcagtgtggtgctcaccttgggcacatttttgatgatgggcctcgtccaactgggaaaagatactgcataaattcggctgccttgtcttttacacctgcggatagcagtggcaccgccgagggaggcagtggggtcgccagcccggcccaggcagacaaagcggagctctagagtaatggagagtgatggaaacaaagtgtacttaatgcacagcttattaaaaaaatcaaaattgttatcttaatagatatattttttcaaaaactataagggcagttttgtgctattgatattttttcttcttttgcttaaacagaagccctggccatccatgtattttgcaattgactagatcaagaactgtttatagctttagcaaatggagacagctttgtgaaacttcttcacaagccacttataccctttggcattcttttctttgagcacatggcttcttttgcagtttttccccctttgattcagaagcagagggttcatggtcttcaaacatgaaaatagagatctcctctgcagtgtagagaccagagctgggcagtgcagggcatggagacctgcaagacacatggccttgaggcctttgcacagacccacctaagataaggttggagtgatgttttaatgagactgttcagctttgtggaaagtttgagctaaggtcatttttttttttctcactgaaagggtgtgaaggtctaaagtctttccttatgttaaattgttgccagatccaaaggggcatactgagtgttgtggcagagaagtaaacattaccacactgttaggcctttattttattttattttccatcgaaagcattggaggcccagtgcaatggctcacgcctgtgatcccagcactttgggaggccaaggcgggtggatcacgaggtcaggagatggagaccatcctggctaacatggtgaaaccccgtctctactaaaaatacgaaaaattagccaggcgtggtggtgggcacctgtagtcccagctactcaggaggctgaggcaggagaatggcgtgaacccggaaggcggagcttgcagttagccgagatcatgccactgcactccagcctacatgacaatgtgacactccatctcaaaaaataataataataacaatataagaactagctgggcatggtggcgcatgcatgtagtcccagctactcctgaggctcagtcaggagaatcgcttgaacttgggaggcggaggttgcagtgagctgagctcataccactgcactccagcctgaacagagtgagatcctgtcaaaaaagaaaagaaaaagaaagcagcattcaaatgtaagacaactgtaaaatattgagccccacttggtctaaaattcaaaaagaagaacgcctgtccatcgcctttttataagtccttctctccacacctaaaagcagctgcagctggaagggcacaaattccactgtgtaaaataaaatattaggggcaacacacttcatcaaggcagcaggaatgagagagagcagagaagatcaaggatgaagtcttgggtactgaaaaattcagtgctgggcagaaaaactgacagggcagtacaagtaacaaacagaatccaagtggggtggcccttgtgcacagagctccaggtgacctctggagagacatgggcattcacatggaaagctaaaacggaagctcaagtttcatactcaacataatcttctgtgtgacaaaggacaagccatgtagcctctctgtgcctatttcttcatgcataaactgggactcataatatttgtaaaatgtattgatactctcagggcaaattcactatattgctatacagttgagatcagtgttgtaaaattaaactgatctggttctaattgcctcaaaggccaaagcccaggcatttgaaatggaaagaagcagagaggaggctgacttagctgattggtatggaaacagttgggccaagagccagaatttccctttgtagcaacacggctagttttactttgagaagctctgctcagctgctttataacattaagtctggcggaatggatgtcactgtgcacaataaagttttcacaagtataaacaatggtgatgtaagtcaacattgctgtagccaggtgtgaaggttgtatggtgtgtgacgaatgtacatcatgtttgtaggtttggatgctaatcttgaattgtagtttaaaaaatacgtatttttgtaactctttgaaagtttatgaagactgacagctttccttgtaagcactaagagaaaaaaaagaaagagggacatttgacaattttaaagaaacaacaagaaattagaatgaaaatctgtgacaaacagcgtcagtgtggccatgtccacattcctacatgtctctctctacaagcacctctctaagaagcctgacatcccggtggactctttatagtcatgtacacttgattccagatgagctctggtcttatctggatgctcagataagaggtttctatctgagcatccagatgttccctcaggttccaagacatttcaccccaggccctgggttcactctggaattcgtaggcttcacgtctctctagaaatgacgtgtaaaatttaagaccagacctcagccatcagcgtccagaccatcctagaagtctttcccaatctcacagagaaagccctagtatttcccagtgaccccaggattccacgttggggtggccaaagaaataggtctctcagggctttgccacagcctccagcccatccttcagaggcacacacagcacctctcggctgctccagctctgtaggatagcctcccctggggtccgtgggacgcgggccacagtgttgaggtagacaaggaggatcagtgagaggcctcttccctctccacagagactggattgtcattgttccttcatttatatcgtagggcttaacatttcactcaaaaaaaagcccctctttttctaatccttagtctttgtttcaaggaaagccagtttttcttctaccacattttccaggatcgactttaagaaaaatgcaacatctattgaaaaaaagtggggtgtatgcatgtggtttaattccagattgcttttgggtttaagtggtatcaaatttcagtatatttctgtcttatgtgaaagaaatatattactaaaacgtcagtgagcaataatgtcagctgtcaagcactagatttatttttgcaggatatggagtgcaatgaactgagtcaatatggcaaggtgtatgtgatctgtgggagttatgccatttaacataggaagtgcatgggactttccctctctgcactccagctcttactgtaccattagaagatgcagaattctgttggtgtgcaaaaagtatagccttacattcaagcagaatggatctgaagaaagcagcaatatctgttactagagaacattcccatgtgtttaaactcttcacttcttagatgcatttaaattcttaatgcaaatgacgtagcaatttgaaaacttctccgtattacttgtgtttaaaatgtcttgctttaaatacaaaacaaatggtaaaggggattatcttttgtttagatggttaaatattatttttgccttagatagctttgtaataatttttctccagacagttcaacacttttgaaaaatgacatgaattttcattaaaaacccttttcctatgtttattgtatacaagaattatgcaataaaatttctttataaaaataaaaaaaaaaaaaaaaaa

SEQ ID NO: 20 - protein, 185 aa, Homo sapiens; identical to SEQ ID NO: 16